(19) United States
(12) Patent Application Publication
(10) Pub. No.: US 2004/0092013 A1
(43) Pub. Date: May 13, 2004

(54) Method of treating alzheimer's disease with cell therapy

(76) Inventors: Evan Y. Snyder, La Jolla, CA (US); Jeanne F. Loring, Del Mar, CA (US); Gary L. Snable, Atherton, CA (US); Karen S. Aboody, Arcadia, CA (US); Marcel M. Daadi, Palo Alto, CA (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/344,712

(22) PCT Filed: Aug. 14, 2001
(86) PCT No.: PCT/US01/25629

(51) Int. Cl. A61K 45/00; C12N 5/08

(52) U.S. Cl. 435/368; 424/93.7

(57) ABSTRACT

A method of treating Alzheimer's disease provides for administering NSC to a susceptible individual. Preferably the NSCs are administered intracisternally. Other administration routes are spinal injection, ventricular injection or systemic injection. Preferably, the quantity of NSC administered is in a range of about 400,000 to about 40,000,000. More preferably, the quantity of NSC is about 1,000,000 to about 10,000,000. The NSCs are administered at multiple locations. The NSCs can be administered to the neocortex or other affected areas of both hemispheres. The method of preventing further deterioration in cognitive function in a person diagnosed with Alzheimer's disease provides for administering NSC to the person in sufficient quantity to prevent additional loss of cognitive function.

### PRIOR APPLICATIONS

This application claims priority to U.S. patent application Ser. No. 60/225,317 filed Aug. 14, 2000, and PCT US01/25629 filed on Aug. 14, 2001.

### TECHNICAL FIELD

This invention is in the field of cell therapy. More particularly, this invention provides a new model for Alzheimer's disease and a method of treating Alzheimer's disease with human stem and neuronal cells.

### BACKGROUND

AD is due to a degenerative process characterized by progressive loss of cells from the basal forebrain, cerebral cortex and other brain areas. Acetylcholine-transmitting neurons and their target nerves are particularly affected, although the lost cells produce a variety of neurotransmitters. Senile plaques and neurofibrillary tangles are present. Pick's disease has a similar clinical picture to AD but a somewhat slower clinical course and circumscribed atrophy mainly affecting the frontal and temporal lobes.

Analysis of the post-mortem brains of Alzheimer's disease victims has led to several theories about the underlying causes of pathology. Prominent among these theories are 1) chronic inflammation (Eikelenboom and Verhius. Exp gerontol 34: 453-61, 1999; Gahtan and Overmier, Neurosci Biobehav rev 272: 32321-3238, etc.), 2) oxidative stress (Smith et al. J Neurosci 17:2653-2657, 1997), 3) mitochondrial defects (Beal. Biochim Biophys Acta 1366: 211-23, 1998), 4) aberrant phosphorylation (Trojanowski and Lee. Am J Path 144: 449-53, 1994), 5) and inappropriate cell cycle signals (Raina et al. Int J Exp Pathol 80:71-6, 1999). The diversity of theories and proposed mechanisms indicates that AD may involve multiple physiological changes.

Animal models for AD and other dementias display hereditary tendency toward the formation of such plaques. Transgenic animal lines have been produced, including two human genes known to be involved in AD pathology: amyloid precursor protein (APP) and the E4 isoform of apolipoprotein E (apoE4). The mutated human APP gene also was added to the transgenic animal. It is thought that if a drug has an effect in the model, it also may be beneficial in at least some forms of AD and Pick's disease. At present there are palliative treatments but no means to restore function.

A group of degenerative disorders characterized by progressive ataxia is due to degeneration of the cerebellum, brainstem, spinal cord and peripheral nerves, and occasionally the basal ganglia. Many of these syndromes are hereditary; others occur sporadically. The spinocerebellar degenerations are logically placed in three groups: predominantly spinal ataxias, cerebellar ataxias and multiple-system degenerations. To date there are no treatments. Friedrich's ataxia is the prototypical spinal ataxia whose inheritance is autosomal recessive. The responsible gene has been found on Chromosome 9. Symptoms begin between ages of 5 and 15 with unsteady gait, followed by upper extremity ataxia and dysarthria. Patients are reflexic and lose large-fine sensory modalities (vibration and position sense). Two other diseases have similar symptoms: Bassen-Kornzweig syndrome (αβ lipoproteinemia, vitamin E deficiency) and Refsom's disease (phytanic acid storage disease). Cerebellar cortical degenerations generally occur between ages 30 and 50. Clinically only signs of cerebellar dysfunction can be detected, with pathologic changes restricted to the cerebellum and occasionally the inferior olives. Inherited and sporadic cases have been reported. Similar degeneration may also be associated with chronic alcoholism.

In multiple-system degenerations, ataxia occurs in young to middle adult life in varying combinations with spasticity and extrapyramidal, sensory, lower motor neuron and autonomic dysfunction. In some families, there may also be optic atrophy, retinitis pigmentosa, ophthalmoplegia and dementia.

Neurotransplantation has been used to explore the development of the central nervous system and for repair of diseased tissue in conditions such as Parkinson's and other neurodegenerative diseases. The experimental replacement of neurons by direct grafting of fetal tissue into the brain has been accomplished in small numbers of patients in several research universities; but so far, the experimental grafting of human fetal neurons has been limited by scarcity of appropriate tissue sources, logistic problems, legal and ethical constraints and poor survival of grafted neurons in the human host brain.

A source of implantable neurons, which is the most ethically controversial, is that of human fetal tissue. U.S. Pat. No. 5,690,927 issued Nov. 25, 1997, also utilizes human fetal tissue. Human fetal neuro-derived cell lines are implanted into host tissues. The methods allow for treatment of a variety of neurological disorders and other diseases. A preferred cell line is SVG.

U.S. Pat. No. 5,753,491 issued May 19, 1998, is an invention that generally relates to methods for treating a host by implanting genetically unrelated cells in the host. More particularly, the present invention provides human fetal neuro-derived cell lines, and methods of treating a host by implantation of these immortalized human fetal neuro-derived cells into the host. One source is the mouse, which is included in the U.S. Pat. No. 5,580,777 issued Dec. 3, 1996. This patent encompasses a method for the in vitro production of lines of immortalized neural precursor cells, including cell lines having neuronal and/or glial cell characteristics, comprises the step of infecting neuroepithelium or neural crest cells with a retroviral vector carrying a member of the myc family of oncogenes.

U.S. Pat. No. 5,753,506 issued May 19, 1998 reveals an in vitro procedure by which a homogeneous population of multipotential precursor cells from mammalian embryonic neuroepithelium (CNS stem cells) was expanded up to 10⁹ fold in culture while maintaining their multipotential capacity to differentiate into neurons, oligodendrocytes, and astrocytes. Chemically defined conditions are presented that enable a large number of neurons, up to 50% of the expanded cells, to be derived from the stem cells. In addition, four factors—PDGF, CNTF, LIF, and T3—have been identified which, individually, generate significantly higher proportions of neurons, astrocytes, or oligodendrocytes. These defined procedures permit a large-scale preparation of the mammalian CNS stem cells, neurons, astrocytes, and oligodendroctytes under chemically defined conditions with efficiency and control. These cells are proposed as an important tool for many cell- and gene-based therapies for neurological disorders.

Another source of stem cells is that of primate embryonic stem cells. U.S. Pat. No. 5,843,780 issued Dec. 1, 1998, utilizes these stem cells. A purified preparation of stem cells is disclosed. This preparation is characterized by the following cell surface markers: SSEA-1 (−); SSEA-3 (+); TRA-1-60 (+); TRA-1-81 (+); and alkaline phosphatase (+). In one embodiment, the cells of the preparation have normal karyotypes and continue to proliferate in an undifferentiated state after continuous culture for eleven months. The embryonic stem cell lines also retain the ability, throughout the culture, to form trophoblasts and to differentiate into all tissues derived from all three embryonic germ layers (endoderm, mesoderm and ectoderm). A method for isolating a primate embryonic stem cell line is also disclosed in the patent.

Yandava et al (PNAS USA 95:7029-34, 1999) demonstrated that neural stem cells implanted at birth resulted in widespread engraftment throughout the shiverer mouse with oligodendrocytes capable of myelinating up to 52% of host neuronal processes with better compacted myelin of a thickness and periodicity more closely approximating normal. Some mice even shivered less.

PCT Publication WO 00/64459 to Layton Bioscience discloses use of LBS neurons in patients who have undergone a stroke. Even at the low doses used in a safety study, some improvement was noted.

In summary, there is substantial evidence in both animal models and human patients that neural transplantation is a scientifically feasible and clinically promising approach to the treatment of focal neurodegenerative diseases, such as Parkinson's disease. Nevertheless, alternative cell sources and novel strategies for differentiation are needed to circumvent the numerous ethical and technical constraints that now limit the widespread use of neural transplantation.

### SUMMARY OF THE INVENTION

A method of treating Alzheimer's disease includes administering NSC to a susceptible individual. A method of treating Alzheimer's disease in an individual diagnosed therewith includes administering NSC to the individual. Preferably, the NSCs are administered parenterally. Parenteral administration may be by spinal tap, ventricular injection or systemic injection. Preferably, the quantity of NSCs administered is between about 400,000 and about 40,000,000. More preferably, the NSCs are administered in a quantity between about 1,000,000 and about 10,000,000. Alternately, the NSCs are administered at multiple locations. The NSCs are administered to the neocortex of both hemispheres. More preferably, the NSCs are of a human origin.

Another method provides for preventing further deterioration in cognitive function in a person diagnosed with Alzheimer's Disease; the method includes parenterally administering NSCs to the person in sufficient quantity to prevent additional loss of cognitive function.

### DETAILED DESCRIPTION OF THE INVENTION

This invention calls for the administration of stem cells or neuronal cells to individuals experiencing the effects of CNS neuron loss. The cells can be administered a variety of ways.

Human NSCs have been developed using a combination of epigenetic and genetic procedures for perpetuation of human neural stem cell lines. Various culture conditions and genes for those that optimally allow for the continuous, rapid expansion and passaging of human neural stem cells. Among them, v-myc (the p110 gag-myc fusion protein derived from the avian retroviral genome) seems to be the most effective gene. There also is a strict requirement for mitogens FGF-2 and EFG in the growth medium, in effect constituting a conditional perpetuality or immortalization. The monoclonal, nestin-positive, human neural stem cell line (HNSC.100) perpetuated in this way divides every 40 hr and stops dividing upon mitogen removal, undergoing spontaneous morphological differentiation and upregulating markers of the three fundamental lineages in the CNS (neurons, astrocytes and oligodendrocytes). The NSCs further have the ability to integrate into most CNS structures.

HNSC.100 cells therefore retain basic features of epigenetically expanded human neural stem cells. Clonal analysis confirmed the stability, multipotency, and self-renewability of the cell line. Finally HNSC.100 can be transfected and transduced using a variety of procedures and genes encoding proteins for marking and therapeutic purposes (e.g., human tyrosine hydroxylase I). (Villa A et al. Exp Neurol 161:67-84, 2000).

NSCs have extraordinary migratory capacity that has made them useful in therapies demanding brainwide dissemination. “Global” cell replacement was demonstrated in lysosomal storage disease (Sly disease) (Snyder E Y et al. Nature 374:367-70, 1995) and the dysmyelinated shiverer mouse (lacking myelin basic protein) (Yandava B D et al. PNAS USA 96:7029-34, 1999). Aboody K S et al. reported that NSC also migrated with metastasizing glioblastoma islands and individuals—whether drawn because of inflammation or degeneration (PNAS USA 97:12846-51, 2000).

Analyzing these data we hypothesized that the NSCs would be useful in treating Alzheimer's disease. Several brain areas are damaged in Alzheimer's disease, neurons are lost and inflammation is increased. NSCs migrate to distant areas to replace the lost cortical neurons and as necessary, glial cells and astrocytes. NSCs migrate to amyloid plaques characterized by neurodegeneration and therein replace lost neurons and provide trophic support.

### Definitions

“Neuronal cells” are those having at least an indication of neuronal phenotype, such as staining for one or more neuronal markers. Examples of neuronal markers include, but are not limited to, neuron-specific nuclear protein, tyrosine hydroxylase, and calbindin. Neuronal stem cells are those that can adopt the phenotype of a variety of nerve cells, as well as the phenotype of, for example, astrocytes and dopaminergic cells.

“Non-tumorigenic” refers to the fact that the source has not been known to cause tumors.

### General Methods

Standard molecular biology techniques known in the art and not specifically described are generally followed as in Sambrook et al., MOLECULAR CLONING: A LABORATORY MANUAL, Cold Springs Harbor Laboratory, New York, 1989 and 1992; and in Ausubel et al., CURRENT PROTOCOLS IN MOLECULAR BIOLOGY, John Wiley and Sons, Baltimore, Md., 1989. Polymerase chain reaction (PCR) is carried out generally as in PCR PROTOCOLS: A GUIDE TO METHODS AND APPLICATIONS, Academic Press, San Diego, Calif., 1990. Reactions and manipulations involving other nucleic acid techniques, unless stated otherwise, are performed as generally described in Sambrook, et al., 1989, MOLECULAR CLONING: A LABORATORY MANUAL, Cold Springs Harbor Laboratory Press, and methodology as set forth in U.S. Pat. Nos. 4,666,828; 4,683,202; 4,801,531; 5,192,659; and 5,272,057 and incorporated herein by reference. In-situ PCR in combination with Flow Cytometry can be used for detection of cells containing specific DNA and mRNA sequences (Testoni et al., Blood 87:3822, 1996).

### Immunoassays

Standard methods in immunology known in the art and not specifically described are generally followed as in Stites et al. (eds), BASIC AND CLINICAL IMMUNOLOGY (8^{th} Edition), Appleton & Lange, Norwalk, Conn., 1994; and Mishell and Shigi (eds), SELECTED METHODS IN CELLULAR IMMUNOLOGY, W. H. Freeman and Co., New York City, 1980.

In general immunoassays are employed to assess a specimen such as for cell surface markers or the like. Immunocytochemical assays are well known to those skilled in the art. Both polyclonal and monoclonal antibodies can be used in the assays. Where appropriate other immunoassays, such as ELISAs, radioimmunoassays (RIA) can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al., MOLECULAR CLONING: A LABORATORY MANUAL, Cold Springs Harbor, N.Y., 1989.

### Gene Therapy

Gene therapy as used herein refers to the transfer of genetic material (e.g. DNA or RNA) of interest into a host to treat or prevent a genetic or acquired disease or condition. The genetic material of interest encodes a product (e.g. a protein, polypeptide, peptide, functional RNA, antisense) whose in vivo production is desired. For example, the genetic material of interest can encode a hormone, receptor, enzyme, polypeptide or peptide of therapeutic value. Alternatively, the genetic material of interest encodes a suicide gene. For a review see, in general, the text “Gene Therapy” (ADVANCES IN PHARMACOLOGY 40, Academic Press, 1997).

### Delivery of Cells

The cells of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved cognition, survival rate or more rapid recovery, or improvement or elimination of symptoms and other indicators as are selected as appropriate measures by those skilled in the art.

In the method of the present invention, the cells of the present invention can be administered in various ways as would be appropriate to implant in the central nervous system. The NSC can be administered intracisternally, parenchymally, intravenously, intraventricularly or by other convenient means.

The human NSC dose with which to alleviate Alzheimer-like symptoms in an animal model is in the range of about 20,000 cells to about 500,000 cells. For a primate test, the dose is about 150,000 to 2,000,000 cells; and for humans, the NSC dose is in the range of 500,000 to 60,000,000 cells. The doses can be divided among different locations, particularly areas most affected such as the basal forebrain, cerebral cortex and hippocampus. Additional doses can be given for greater effect.

### EXAMPLES

### Example 1

NSCs (human HNSC.100, or murine H1 and/or C17.2) are implanted into transgenic mice engineered to produce mutant amyloid precursor protein. Examples of such transgenic mice have been disclosed in a number of patents (see above). Particularly preferred for testing is the transgenic mouse disclosed in U.S. Pat. No. 5,898,094. This mouse has enhanced AD related to amyloid accumulation in its brain because it has at least one of both transgenes contributing to Aβ formation: DNA for mutant presenilin and DNA encoding mutant APP. This transgenic mouse has been found to be a more robust AD model than parent mice having either gene alone; it produces earlier plaques (visible at 15 weeks and full-blown at 26-32 weeks) and earlier behavioral changes than prior models (at 12-14 weeks). Differences were particularly evident in both total number of arm entries (higher) and the spontaneous alternation behavior in the symmetrical “Y” maze (lower). This mouse will be referred to as Tg APP/PS1.

Another transgenic model (referred to below as Tg APP) for AD is that of U.S. Pat. No. 6,046,381 invented by Lennart Mucke et al. and assigned to the University of California. These apoE^{-/-} knockout mice do not produce mouse apoE. Some were transformed with transgenes to produce human apoE3 or apoE4. The apoE^{-/-} and apoE4-transformed mouse brain had age-related changes similar to a human diagnosed with AD, namely loss of presynaptic terminals and neuronal dendrites in the neocortex and hippocampus. Behavioral changes also correlated with apoE^{-/-} and apoE4 transformation; however, there was a gender difference: females took a significantly longer time to locate a hidden, submerged platform in a water maze. Females also had impaired passive avoidance learning, continuing to reenter a dark compartment and receive a shock.

The NSCs used in this experiment are NSC 17.2 murine NSC line and H1 human NSC line; both have the β-galactosidase reporter gene and are neo (G418) resistant. NSCs (3 μl of 100,000 cells/μl) are administered into the right and left ventricles of three control mice and three each of the Tg APP and Tg App/PS1 mice, all 14 months old. The stereotaxic coordinates for both AP: −0.9; LM: ±1.0; DV: −2.5. The mice are tested for behavioral effects at 12 and 32 weeks, just prior to sacrifice. After sacrifice, standard techniques are used to cryopreserve the brain. One hemisphere of each brain is gently shaken in 0.9% saline for 30 minutes and immersion-fixed in 4% paraformaldehyde for 24 hours, and cryoprotected in 15% sucrose/PBS followed by overnight immersion in 30% sucrose/PBS.

Known techniques are used to stain for amyloid deposits and can include: primary antibodies to AB residues 17-24 (4G8, Senetek, Maryland Heights, Mo., 1:1000), GFAP (Boehringer Mannheim, Indianapolis, Ind., 1:1000); counterstaining with Congo red and evaluations using cross-polarized illumination; and staining with 1% thioflavin S after 10 min in Mayer's hematoxylin to mask nuclear fluorescence. NSCs are identified by the presence of lac-Z and by antibodies specific only to human epitopes, including anti-human mitochondria (hMit), anti-human nuclei (hNuc; Chemicon), and anti-human nuclear matrix antigen (NuMA; Calbiochem). The differentiation fate of the engrafted NSCs also will be determined.

### Example 2

Isolated NSCs with migratory capacity are implanted either in the cerebral ventricles or in the parenchyma of diseased CNS area. Suspension of hNSCs (3 μl of 100,000 cells/μl) are administered into the right and left ventricles of control mice and test groups of either the Tg APP and Tg App/PS1 mice, all 14 months old or both. The stereotaxic coordinates for both AP: −0.9; LM: ±1.0; DV: −2.5. NSCs are also deposited at multiple depths and sites of the cerebral cortex and hippocampus of each cerebral hemisphere. The following stereotaxic coordinates are employed: Cortex AP: 1.0; L: ±2.0; DV: −2.5-3.5; Hippocampus: AP: −2.7; L: ±2.5; DV: −3-4. The mice are tested for behavioral effects at 12 and 32 weeks, just prior to sacrifice. After sacrifice, standard techniques are used to cryopreserve the brain. One hemisphere of each brain is gently shaken in 0.9% saline for 30 minutes and immersion-fixed in 4% paraformaldehyde for 24 hours, and cryoprotected in 15% sucrose/PBS followed by overnight immersion in 30% sucrose/PBS.

Known techniques are used to stain for amyloid deposits and can include: primary antibodies to AB residues 17-24 (4G8, Senetek, Maryland Heights, Mo., 1:1000), GFAP (Boehringer Mannheim, Indianapolis, Ind., 1:1000); counterstaining with Congo red and evaluations using cross-polarized illumination; and staining with 1% thioflavin S after 10 min in Mayer's hematoxylin to mask nuclear fluorescence. NSCs are identified by the presence of lac-Z and by antibodies specific only to human epitopes, including anti-human mitochondria (hMit), anti-human nuclei (hNuc; Chemicon), and anti-human nuclear matrix antigen (NuMA; Calbiochem). The differentiation fate of the engrafted NSCs also will be determined.

### Example 3

In a new way of analyzing the difference between Alzheimer's patients and other elderly persons, postmortem samples were subjected to expression profiling on Incyte Unigene Lifearray microarrays (Incyte Genomics, Inc., Palo alto, Calif.). The portmortem samples were collected fresh at autopsy and flash frozen in isopentane at −60° C. and stored at −80° C. After fixing and staining, numbers of diffuse and neuritic core plaques and neurofibrillary tangles were counted in a 200× high power field. The diagnosis of AD was based on CERAD criteria. Control cases had no neurological and/or psychiatric history.

The microarrays had approximately 7050 hybridizable sequences; the detectable signals from brain tissue were 2486. A total of 910 mRNA were differentially expressed in brain and 118 were differentially expressed AD amygdala and cingula. The controls were used to “subtract out” electronically the array of genes expressed in controls in extremis. The final group was limited to mRNA dependably expressed in one group and not the other. In the AD group, there were increases in cell adhesion, proliferation and inflammation-related mRNA. There were decreases in mRNA for signal transduction, secretory vesicles and stress/energy metabolism. There also were 10 new mRNA of unknown function, which correlated with the pathology.

These findings support the several theories of AD dysfunction: chronic inflammation, oxidative stress, mitochondrial defects, proteasome dysfunction, aberrant phosphorylation and inappropriate cell cycle signals.

These findings represent end-stage AD, and do ot provide information on the start of the process. However, definitive therapy, such as cell transplantation, will not be undertaken until the AD patient shows significant symptoms. To help improve the defects broadly painted by the gene expression profile, NSCs are administered to replace neurons and brain architectue; NSCs can spread widely throughout the affected areas; NSCs also can develop into glial and other cells to improve the brain architecture. LBS neurons also can be used to replace AD-affected neurons or can be administered with NSCs. The NSCs can be administered intravenously, intrathecally, intraventricularly, intracisternally, or by other convenient methods.

The foregoing description and examples are intended only to illustrate, not limit, the disclosed invention.

We claim:

1. A method of treating Alzheimer's disease comprising administering NSC to a susceptible individual.

2. A method of treating Alzheimer's disease in an individual diagnosed therewith, the method comprising administering NSC to the individual.

3. The method of claim 1 wherein the administering of the NSC is done by administering the cells parenterally.

4. The method of claim 3 wherein the parenteral administration is by spinal tap, ventricular injection or systemic injection.

5. The method of claim 1 wherein the NSC are administered in a quantity between 400,000 and 40,000,000.

6. The method of claim 5 wherein the NSC are administered in a quantity between 1,000,000 and 10,000,000.

7. The method of claim 1 wherein the NSC are administered at multiple locations.

8. The method of claim 7 wherein the NSC are administered to the neocortex of both hemispheres.

9. A method of preventing further deterioration in cognitive function in a person diagnosed with Alzheimer's Disease, the method comprising parenterally administering NSC to the person in sufficient quantity to prevent additional loss of cognitive function.

10. A method of targeting therapeutic genes to the Alzheimer's-affected brain regions, comprising administering NSC with a therapeutic gene to an individual with Alzheimer's disease.
